(19) United States
(12) Patent Application Publication
(10) Pub. No.: US 2009/0073718 A1
(43) Pub. Date: Mar. 19, 2009

(54) SEPARABLE MEDICAL ILLUMINATING DEVICE

(76) Inventor: Donny CHUNG, Taipei (TW)

Correspondence Address: HDLS Patent & Trademark Services, P.O. BOX 220746, CHANTILLY, VA, 20153-0746, US

(21) Appl. No.: 11/856,798

(22) Filed: Sep. 18, 2007

(51) Int. Cl. A61B 1/06; G02B 6/36

(52) U.S. Cl. 362/572; 385/92

(57) ABSTRACT

A separable medical illuminating device includes a light source transmitting assembly and a grip assembly. The light source transmitting assembly is constituted of a light source transmitting body, a protective body and a connecting body. The light source transmitting body is constituted of an optical fiber and a covering body. The protective body is connected to one end of the light source transmitting body. The connecting body allows an end of the light source transmitting body to be inserted therein. The grip assembly is constituted of a cylinder, a light source model and an adapter. The light source module is disposed at the front end of the cylinder. After the adapter is assembled with the front end of the cylinder, the light source module is positioned between the front end of the cylinder and the adapter. The connecting body of the light source transmitting assembly is inserted onto the adapter. When the light source module is lighted up, the light is transmitted via the light source transmitting body, and is projected to a predetermined position after being condensed by the protective body.

### BACKGROUND OF THE INVENTION

1. Field of the Invention

The present invention relates to a separable illuminating device, and in particular to a hand-held separable medical illumination device.

2. Description of Prior Art

Medical instruments have to be combined with some advanced technologies such as micro mechanical and electrical technology, communication technology, material technology and bio-chemical technology to form another industrial technology. Therefore, the development of each kind of technique or technology can promote the industry of medical instruments. Owing to the improved medical instruments, the possibility of the success in the medical treatment can be also increased.

The illuminating device used in a traditional medical treatment can be mounted on a ceiling or wall (such as an operation lamp), and alternatively, can be attached to the head of medical personnel. When the medical personnel perform a medical treatment for a patient, the illuminating device is used to illuminate the position of the wound on the body of the patient, so that the medical personnel can see the wound of the patient clearly. In addition to the above two kinds of illuminating devices, another kind of illuminating device currently used by the medical personnel is a hand-held illuminating device. The volume of such kind of hand-held illuminating device is small, so that it can be used to illuminate the wound of the patient if the above two kinds of illuminating devices cannot work in some instances.

However, as shown in FIG. 1, the conventional hand-held medical illuminating device has a grip 10. The interior of the grip 10 is provided with a battery 20, and the front end thereof is assembled with a tube body 30. The front end of the tube body 30 is provided therein with a light-emitting source 40. The light-emitting source 40 is electrically connected with a switch 60 and the battery 20 via electricity-delivering leads 50. In use, the medical personnel only presses the switch 60, the electricity is delivered from the battery 20 to the light-emitting source 40 via the electricity-delivering leads 50, thereby lighting up the light-emitting source 40. In this way, the medical personnel can see the wound of the patient clearly.

Since the tube body 30 of the conventional hand-held medical illuminating device is made of a hard metallic material, it is not easy for the medical personnel to adjust the angle or orientation of the illuminating device. Therefore, the light emitting by the illuminating device may be obstructed by some mussel tissue, which results in a fact that the side or corner of the wound of the patient may not be seen and thus generates a trouble for the medical personnel in performing the medical treatment. Further, since the light-emitting source 40 is fixedly connected and thus integrally formed with the interior of the tube body 30, the tube body 30 cannot be replaced upon the damage of the light-emitting source 40. Therefore, the whole set of the hand-held medical illuminating device has to be discarded, causing the waste of medical resources.

### SUMMARY OF THE INVENTION

In view of the above drawbacks, the present invention provides a separable medical illuminating device. With this arrangement, a light source transmitting assembly and a grip assembly of the medical illuminating device can be separated from each other, thereby replacing the light source transmitting assembly. After the light source transmitting assembly and the grip assembly are separated from each other, they can be stored conveniently. Further, the light source transmitting assembly allows the medical personnel to adjust the angle and orientation of the illuminating device, so that the medical personnel can see every corner of the wound of the patient clearly.

The present invention is to provide a separable medical illuminating device, which includes a light source transmitting assembly and a grip assembly assembled with the light source transmitting assembly. The light source transmitting assembly is constituted of a light source transmitting body, a protective body and a connecting body. The light source transmitting body is constituted of an optical fiber and a covering body. The protective body is provided thereon with a conical body. The front end of the conical body has a curved surface and the rear end thereof is provided with a connecting hole. The connecting hole is connected to one end of the light source transmitting body. The connecting body is provided therein with a penetrating hole that allows the other end of the light source transmitting body to be inserted therein. The grip assembly is constituted of an elongated cylinder, a light source model and an adapter. The cylinder is provided therein with an accommodating space. The accommodating space is disposed with a lead therein. The outer peripheral surface of an opening of the front end of the cylinder is provided with a threaded surface. The light source module is disposed in the opening at the front end of the cylinder, and is provided with a base body and a light-emitting unit arranged on the base body. The light-emitting unit has a light-emitting diode. The base body is provided with a first electrode and a second electrode. The first electrode and the second electrode are electrically connected with the light-emitting diode. The adapter is provided thereon with an assembling port that allows the connecting body to be inserted therein. A tube body extends from one end of the assembling port. A skirt body extends from one end of the tube body. The interior of the skirt body is in communication with the tube body. The inner wall face of the skirt body is provided with a threaded surface. When the threaded surface is threadedly connected with the threaded surface of the front end of the cylinder, the light source model can be assembled between the opening of the front end of the cylinder and the interior of the skirt body, so that the light-emitting diode can be positioned to correspond to the tube body.

### BRIEF DESCRIPTION OF THE DRAWINGS

FIG. 1 is a schematic view showing a hand-held mechanical illuminating device of prior art;

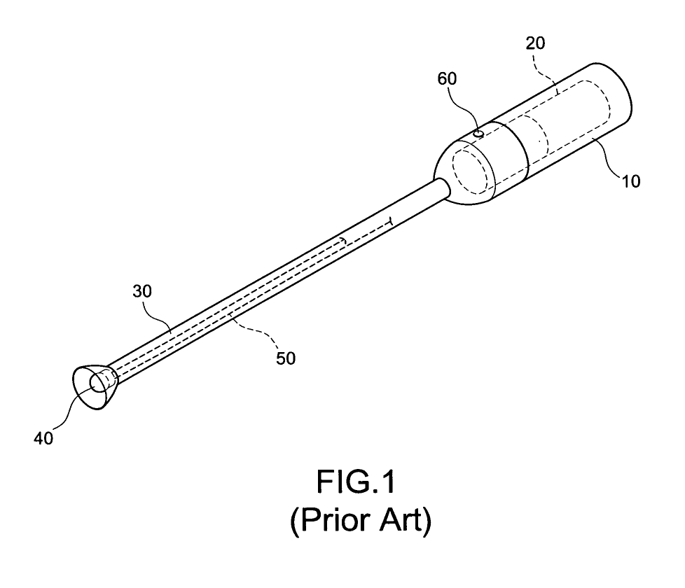

FIG. 2 is an exploded view showing the structure of the light source transmitting assembly of the present invention;

FIG. 3 is an exploded view showing the separable medical illuminating device of the present invention;

FIG. 4 is an exploded view showing the structure of the grip assembly of the present invention seen from another viewing angle;

FIG. 5 is an assembled view showing the separable medical illuminating device of the present invention;

FIG. 6 is a cross-sectional view showing the separable medical illuminating device of the present invention;

FIG. 7 is a cross-sectional view showing the structure of the light source transmitting assembly of the present invention;

FIG. 8 is a schematic view showing an exemplary application of the separable medical illuminating device of the present invention; and

FIG. 9 is a schematic view showing the separable medical illuminating device of another embodiment of the present invention.

### DETAILED DESCRIPTION OF THE INVENTION

The technical contents and the detailed description of the present invention will be explained with reference to the accompanying drawings.

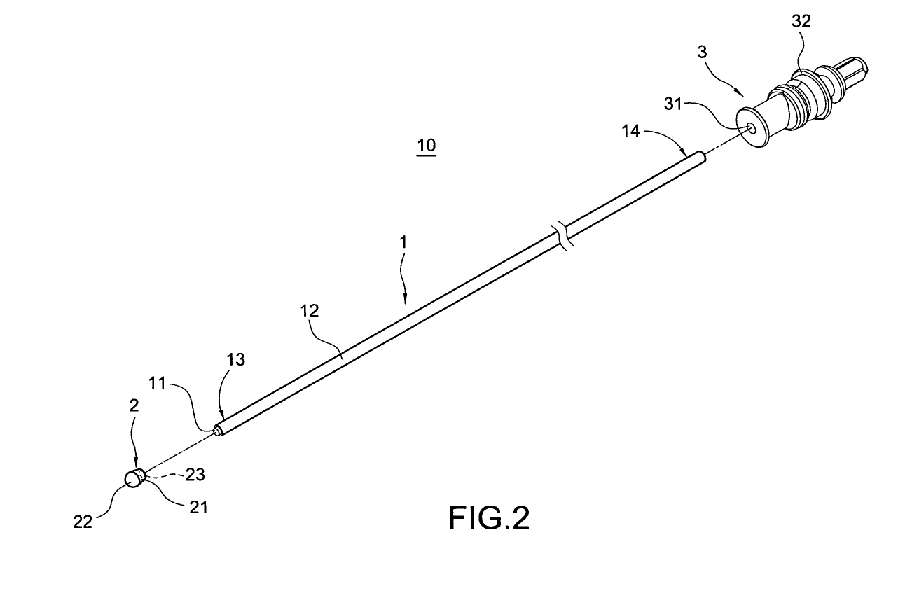

With reference to FIG. 2, it is an exploded view showing the structure of the light source transmitting assembly of the present invention. As shown in this figure, the light source transmitting assembly 10 shown in this figure includes an elongated light source transmitting body 1, a protective body 2 assembled with an end of the light source transmitting body 1, and a connecting body 3 assembled with another end of the light source transmitting body 1.

The light source transmitting body 1 is constituted of a linear transparent optical fiber 11 and a covering body 12 covering on the outer surface of the optical fiber 11. The light source transmitting body 1 is provided thereon with a first end 13 and a second end 14. The light source transmitting body 1 is used to transmit a light source (light source generating end) from the second end 14 to the first end 13, thereby providing an illumination. The covering body 12 prevents the light from leaking during the transmission of the light source. The covering body 12 is made of a flexible metallic material. The covering body 12 covers on the optical fiber 11, thereby adjusting the illuminating direction of the optical fiber.

The protective body 2 is made of a transparent material and has a conical body 21 thereon. The front end of the conical body 21 is provided with a curved surface 22, and the rear end thereof is provided with a connecting hole 23. The connecting hole 23 is used to connect on the first end 13 of the light source transmitting body. The protective body 2 can prevent the sharp corner of the first end 13 from hurting the wound of the patient. Further, the protective body 2 is used to condense or scatter the light transmitted by the light source transmitting body 1 onto a predetermined position.

The connecting body 3 is provided therein with a penetrating hole 31 that allows the second end 14 of the light source transmitting body 1 to be inserted therein. The connecting body 3 is provided thereon with a projecting ring 32. When the connecting body 3 is assembled with the grip assembly (not shown), the projecting ring 32 blocks the front end of an adapter of the grip.

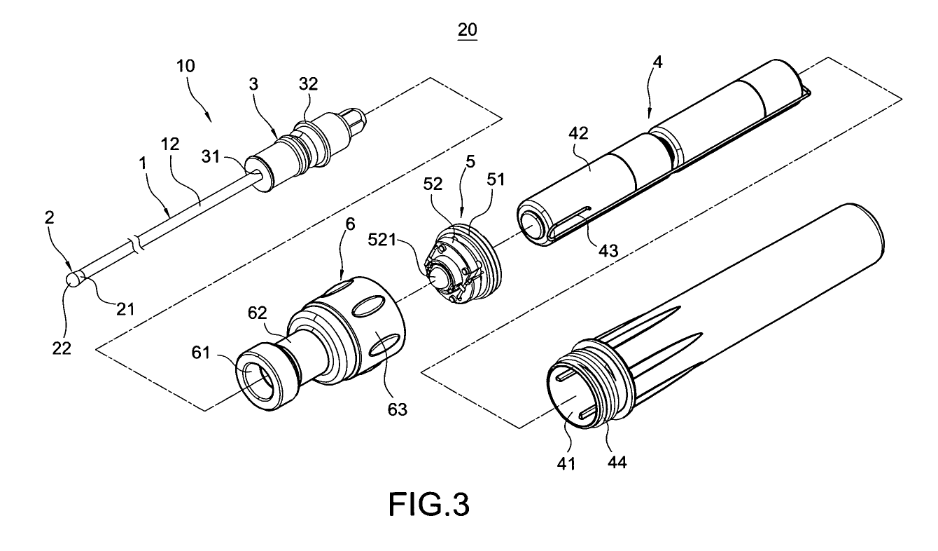

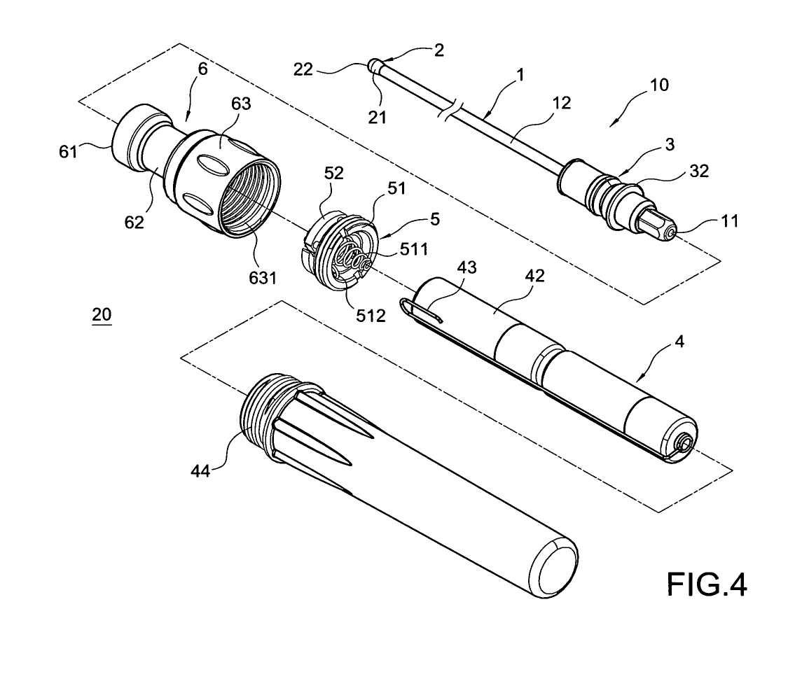

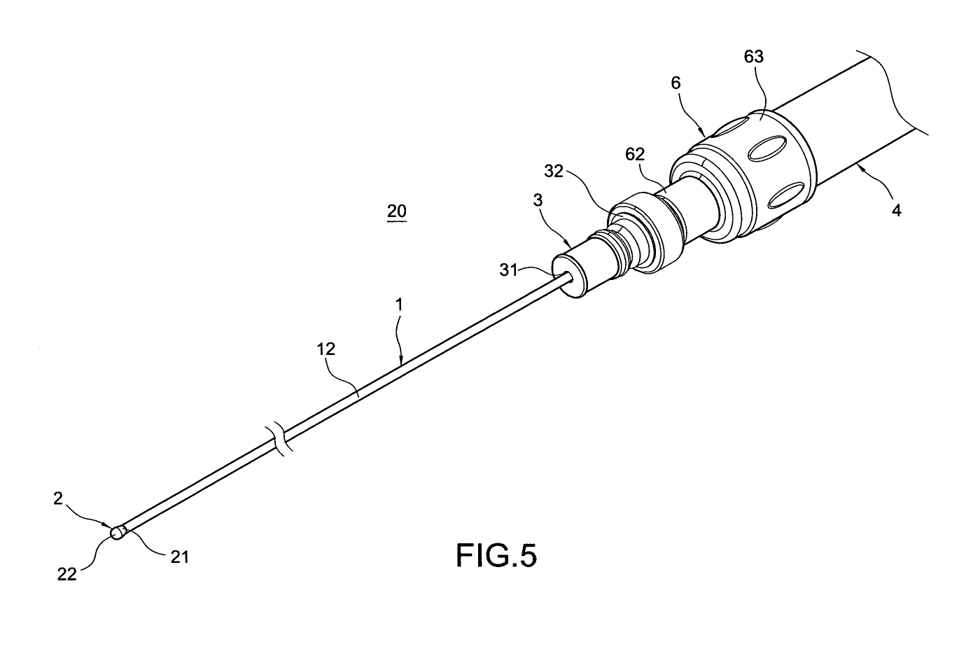

FIG. 3 is an exploded view showing the separable medical illuminating device of the present invention. FIG. 4 is an exploded view showing the structure of the grip assembly of the present invention seen from another viewing angle. FIG. 5 is an assembled view showing the separable medical illuminating device of the present invention. As shown in these figures, the separable medical illuminating device shown in these figures includes a light source transmitting assembly 10 and a grip assembly 20 for allowing the light source transmitting assembly 1 to be assembled therewith.

The light source transmitting assembly 10 is constructed as that mentioned in the previous paragraphs.

The grip assembly 20 is constituted of an elongated cylinder 4, a light source module 5 and an adapter 6.

The cylinder 4 is provided therein with an accommodating space 41. The accommodating space 41 is used to accommodate battery 42 and a lead 43 for delivering a power source. The outer peripheral surface of the opening at the front end of the cylinder 4 is provided with a threaded surface 44. The threaded surface 44 is used to connect with the adapter 6.

The light source module 5 is disposed in the opening at the front end of the cylinder 4. The light source module has a base body 51 and a light-emitting unit 52 disposed on the base body 51. The light-emitting unit 52 has a light-emitting diode 521 thereon. The base body 51 is provided with a first electrode 511 and a second electrode 5 12. The first electrode 511 and the second electrode 512 are electrically connected with the light-emitting diode 521.

The adapter 6 has an assembling port 61 that allows the covering body 3 of the light source transmitting assembly 10 to be inserted therein. A tube body 62 extends from one end of the assembling port 61. A skirt body 63 extends from one end of the tube body 62. The interior of the skirt body 63 is in communication with the tube body 62. The inner wall face of the skirt body is provided with a threaded surface 631. When the threaded surface 631 is threadedly connected with the threaded surface 44 of the front end of the cylinder 4, the light source module 5 can be assembled between the opening of the front end of the cylinder 4 and the interior of the skirt body 63, so that the light-emitting diode 521 is positioned to correspond to the tube body 62. When the light source transmitting assembly 10 is assembled with the grip assembly 20, the distal end of the connecting body 3 of the light source transmitting assembly 10 is inserted into the assembling port 61 of the adapter 6. At this time, the projecting ring 32 of the connecting body 3 blocks at the front of the opening of the assembling port 61.

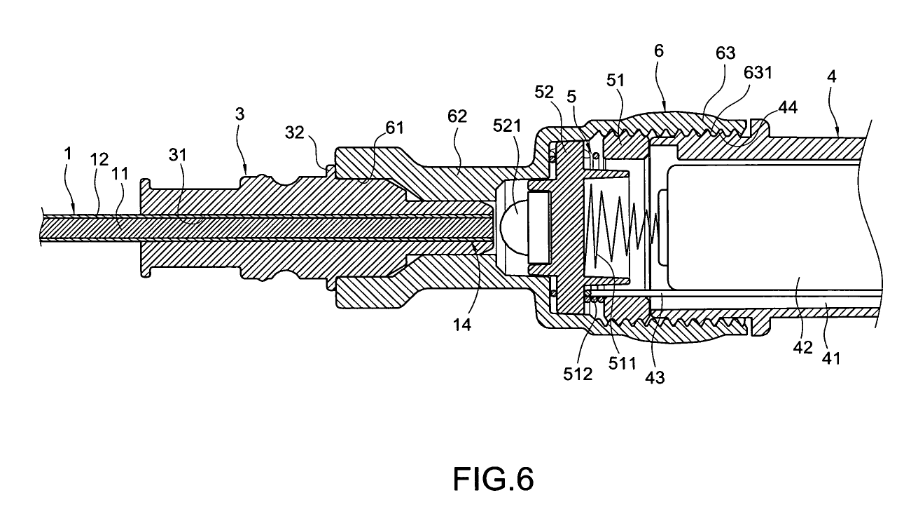

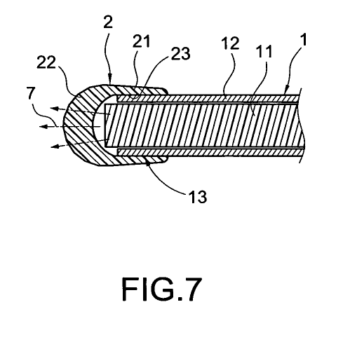

FIG. 6 is a cross-sectional view showing the separable medical illuminating device of the present invention, and FIG. 7 is a cross-sectional view showing the structure of the light source transmitting assembly of the present invention. As shown in these figures, when rotating the adapter 6 or cylinder 4 to cause the movement of the light source module 5, the first electrode 511 at the bottom of the base body 51 of the light source module 5 is brought into contact with a negative pole (or positive pole) of the battery 42, the second electrode 512 is brought into contact with the lead 43, and the other end of the lead 43 is brought into contact with the positive pole (or negative pole) of the battery 42. As a result, the electricity of the battery 42 is delivered to the light-emitting unit 52, thereby lighting up the light-emitting diode 521. The light 7 passes through the tube body 62 of the adapter 6, and is transmitted via the optical fiber 11 of the light source transmitting body 1. When the light 7 is transmitted to the protective body 2, the protective body 2 can condense or scatter the light 7 onto the predetermined position. Further, during a medical treatment, the protective body 2 can protect the sharp corner of the first end 13 of the light source transmitting body 1 from hurting the wound of the patient.

When the light 7 is transmitted in the optical fiber 11, the covering body 12 covering on the outer surface of the optical fiber 11 can prevent the light 7 transmitted by the optical fiber 11 from leaking, so that the light 7 can be transmitted to the protective body 2 efficiently.

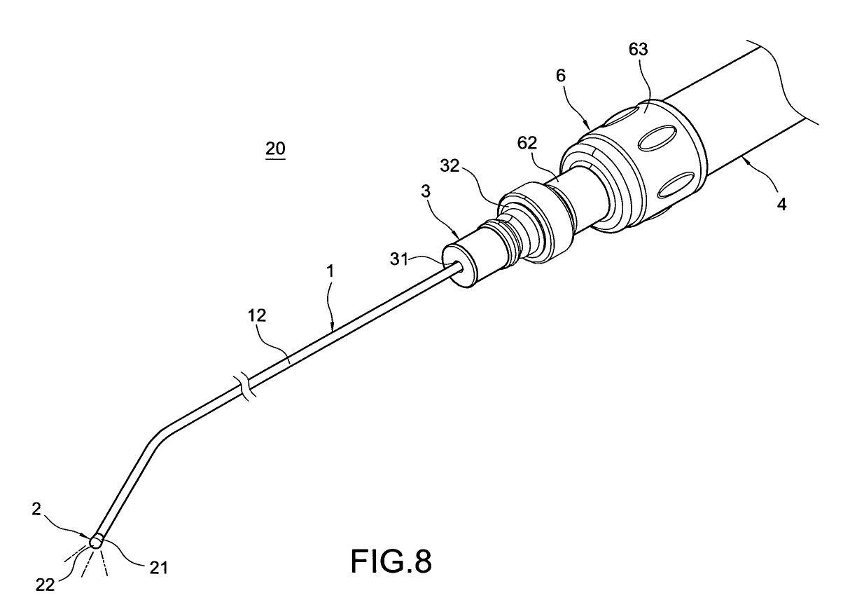

With reference to FIG. 8, it is a schematic view showing an exemplary application of the separable medical illuminating device of the present invention. As shown in this figure, after the light source transmitting assembly 10 and the grip assembly 20 are assembled with each other, the medical personnel can hold the cylinder 4 directly by his/her hand for the subsequent use. In use, if the light projected by the protective body 2 cannot illuminate a certain position of the patient body, the flexible metallic covering body 12 can be used to adjust the angle of illumination, thereby illuminating the predetermined position.

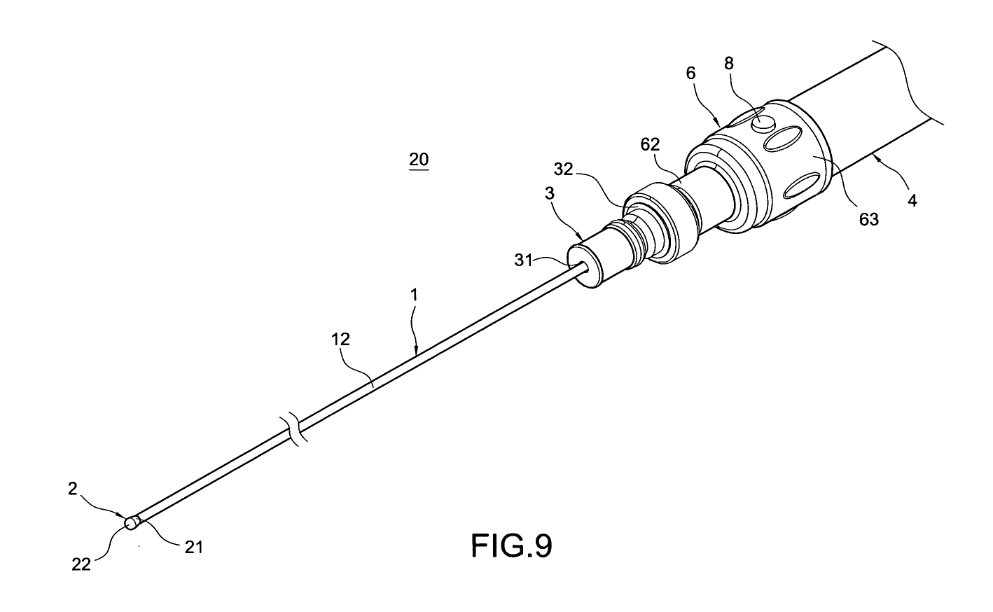

Further, with reference to FIG. 9, a switch 8 can be further provided on the cylinder 4 or the adapter 6. The switch 8 is electrically connected between the light-emitting diode and the battery. The user only needs to press the switch 8. In this way, the electricity of the battery can be delivered to the light-emitting diode, thereby lighting up the light-emitting diode.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. A light source transmitting assembly of a separable medical illuminating device, comprising:
a light source transmitting body including an optical fiber and a covering body covering on an outer surface of the optical fiber, the light source transmitting body being provided thereon with a first end and a second end; and
a protective body assembled on the first end of the light source transmitting body.

2. The light source transmitting assembly of a separable medical illuminating device according to claim 1, wherein the optical fiber is a linear transparent body.

3. The light source transmitting assembly of a separable medical illuminating device according to claim 1, wherein the covering body is made of a flexible metallic material.

4. The light source transmitting assembly of a separable medical illuminating device according to claim 1, wherein the protective body is made of a transparent material and has a main body thereon, a front end of the main body has a curved surface and a rear end thereof is provided with a connecting hole.

5. The light source transmitting assembly of a separable medical illuminating device according to claim 4, wherein the main body of the protective body is formed into a conical shape.

6. The light source transmitting assembly of a separable medical illuminating device according to claim 1, further comprising a connecting body connected onto the second end of the light source transmitting body, the connecting body being provided therein with a penetrating hole, and an outer surface of the connecting body being provided with a projecting ring.

7. A grip assembly of a separable medical illuminating device for assembling with a light source transmitting assembly, the grip assembly comprising:
a cylinder having an opening at a front end thereof, the interior of the cylinder being provided with an accommodating space;
a light source module disposed on the front end of the opening of the cylinder, and having a base body and a light-emitting unit disposed on the base body; and
an adapter assembled with the opening, thereby causing the light source module to be positioned between the interior of the adapter and the opening of the cylinder.

8. The grip assembly of a separable medical illuminating device according to claim 7, wherein a lead for delivering the electricity is arranged in the accommodating space.

9. The grip assembly of a separable medical illuminating device according to claim 7, wherein an outer peripheral surface of the opening at the front end of the cylinder is provided with a threaded surface.

10. The grip assembly of a separable medical illuminating device according to claim 7, wherein the light-emitting unit has a light-emitting diode thereon.

11. The grip assembly of a separable medical illuminating device according to claim 7, wherein the base body is provided with a first electrode and a second electrode, the first electrode and the second electrode are electrically connected with the light-emitting diode.

12. The grip assembly of a separable medical illuminating device according to claim 7, wherein the adapter is provided thereon with an assembling port for allowing the light source transmitting assembly to be connected therein, a tube body extends from one end of the assembling port, a skirt body extends from one end of the tube body, the interior of the skirt body is in communication with the tube body, and the inner wall face of the skirt body is provided with a threaded surface.

13. The grip assembly of a separable medical illuminating device according to claim 7, wherein a switch is additionally provided on one of the cylinder or the adapter, the switch is electrically connected between the light-emitting diode and the battery.
